(19) United States
(12) Patent Application Publication
(10) Pub. No.: US 2008/0085242 A1
(43) Pub. Date: Apr. 10, 2008

(54) NON-VIRAL GENE DELIVERY SYSTEM

(75) Inventors: Per Artursson, Uppsala (NO); Bjorn Erik Christensen, Trondheim (NO); Magnus Koping-Hoggard, Uppsala (NO); Kjell Morten Varum, Trondheim (NO); Kristoffer Tommeraas, Oslo (NO)

Correspondence Address: CONNOLLY BOVE LODGE & HUTZ, LLP, P O BOX 2207, WILMINGTON, DE, 19899, US

(73) Assignee: FMC BIOPOLYMER AS

(21) Appl. No.: 11/848,399

(22) Filed: Aug. 31, 2007

Related U.S. Application Data
(63) Continuation of application No. 10/513,311, filed on Mar. 28, 2005, now abandoned.

(30) Foreign Application Priority Data
May 3, 2002 (NO) 2002 2149

(51) Int. Cl. A61K 31/7088; A61K 49/00; A61P 31/00; A61P 35/00

(52) U.S. Cl. 424/9.1; 514/44

(57) ABSTRACT

The present invention concerns a novel composition comprising a nucleic acid; and a chitosan containing branching groups covalently linked to the amino groups wherein said branches are selected from the following groups; alkyl with 2 or more carbon atoms, monosaccharides, oligosaccharides or polysaccharides. The composition is particularly useful as a non-viral gene delivery system. The composition facilitates the introduction of the nucleic acid into the cells to which it is administrated, as well as the expression of the function of the nucleic acid.

### FIELD OF THE INVENTION

The present invention relates to a new non-viral delivery system for nucleic acids, and more specifically, to a system, which facilitates the introduction of nucleic acid into cells in a host tissue after administration to that tissue. The composition of the present invention is based on the biodegradable polysaccharide chitosan that due to certain chemical modifications achieve more efficient delivery of biologically active nucleic acids, such as oligo- or polynucleotides that encodes a desired product, and/or facilitates the expression of a desired product in cells present in that tissue.

### BACKGROUND OF THE INVENTION

The concept of gene therapy is based on that nucleic acid; DNA or RNA can be used as pharmaceutical products to cause in vivo production of therapeutic proteins at appropriate sites. Delivery systems for nucleic acid are often classified as viral and non-viral delivery systems. Because of their highly evolved and specialised components, viral systems are currently the most effective means of DNA delivery, achieving high efficiencies for both delivery and expression. However, there are safety concerns for viral delivery systems. The toxicity, immunogenicity, restricted targeting to specific cell types, limited DNA carrying capacity, production and packaging problems, recombination and a very high production cost hamper their clinical use (Luo and Saltzman, 2000). For these reasons, non-viral delivery systems have become increasingly desirable in both basic research laboratories and clinical settings. However, from a pharmaceutical point of view, the way of delivery of nucleic acids still remains a challenge since a relatively low expression is obtained in vivo with non-viral delivery systems as compared to viral delivery systems (Saeki et al., 1997).

A variety of non-viral delivery systems, including cationic lipids, peptides or polymers in complex with plasmid DNA (pDNA), have been described in the prior art (Boussif et al., 1995; Felgner et al., 1994; Hudde et al., 1999). The negatively charged nucleic acids interact with the cationic molecules mainly through ion-ion interactions, and undergo a transition from a free form to a compacted state. In this state the cationic molecules may provide protection against nuclease degradation and may also give the nucleic acid-cationic molecule complex surface properties that favour their interaction with and uptake by the cells (Ledley, 1996).

Among these cationic molecules, the synthetic polymer polyethylenimine (PEI) has been shown to form stable complexes with pDNA and mediate relatively high expression of the transgene both in vitro and in vivo (Boussif et al., 1995; Ferrari et al., 1997; Gautam et al., 2001). For this reason, PEI is often used as a reference system in the experimental setup. However, a rough correlation between toxicity and efficiency has been suggested for PEI (Luo and Saltzman, 2000) and recent studies have addressed concerns about toxicity using PEI (Godbey et al., 2001; Putnam et al., 2001). Another drawback with PEI is that it is not biodegradable and it may therefore be stored in the body for a long time. Therefore, the search for effective and non-toxic biodegradable non-viral delivery systems is highly desirable.

Most commonly, non-viral delivery systems have been delivered in vivo by the parenteral route. After intravenous administration to mice, compacted nucleic acid-cationic molecule complexes deposited mainly in the lung capillaries where the gone was expressed in the endothelium of the capillaries in the alveolar septi (Li and Huang, 1997; Li et al., 2000; Song et al., 1997) or even in the alveolar cells (Bragonzi et al., 2000; Griesenbach et al., 1998), but not in the epithelium. However, unformulated, naked DNA was rapidly degraded in the blood circulation before it reached its target and generally resulted in no gene expression. In contrast, injection of naked DNA into skeletal muscle resulted in a dose-dependent gene expression (Wolff et al., 1990) which was further enhanced when complexed with a non-compacting but ‘interactive’ polymer such as polyvinyl pyrrolidone (PVP) or polyvinyl alcohol (PVA) (WO 96/21470) (Mumper et al., 1996; Mumper et al., 1998). Thus, gene transfection in vivo is tissue-dependent in an unpredictable way and therefore remains a challenge.

Mucosal delivery of non-viral delivery systems has also been described, that is delivery to the gastrointestinal tract, nose and respiratory tract (Koping-Hoggard et al. 2001; Roy et al., 1999), WO 01/41810. With exception for the delivery to the nasal tissue where DNA in un-compacted form gives the best gene expression (WO 01/41810) compacted nucleic acid-cationic molecule complexes are preferred to un-compacted DNA when a high gene expression is required in a mucosal tissue.

In prior art, non-viral gene delivery systems are based on cationic polymers (such as chitosan) of rather high molecular weight often several hundred kilodaltons (kDa) with 5 kDa as a lower limit (e.g. Macaughlin et al., 1998; Roy et al., 1999, WO 97/42975). The major reason that polymers of lower molecular weight (<5 kDa) form unstable complexes with DNA, resulting in a low gene expression (Koping-Hoggard, 2001). However, there are many drawbacks using cations of high molecular weight such as increased aggregation of compacted nucleic acid-cationic molecule complexes and solubility problems (MacLaughlin et al., 1998). Further, there are several biological advantages of using cationic molecules of lower molecular weights i.e. they generally show reduced toxicity and reduced complement activation compared to cations of higher molecular weights (Fischer et al., 1999; Plank et al., 1999).

In the prior art some examples of the use of low molecular weight cations for complexation with nucleic acid has been described (Florea 2001; Godbey et al., 1999; Koping-Hoggard, 2001; MacLaughlin et al., 1998; Sato et al., 2001). However, these low molecular weight cations form unstable compacts with DNA that separate in an electric field (agarose gel electrophoresis) resulting in no or a very low gene expression in vitro, as compared to cations of higher molecular weights. This can be explained by that complexes formed between DNA and low molecular weight cations are generally unstable and dissociate easily (Koping-Hoggard, 2001). In fact, the dissociation of cationic molecule-DNA compacts and release of naked DNA during agarose gel electrophoresis has often been used as an assay to distinguish ineffective formulations from effective ones in the literature (Fischer et al., 1999; Gebhart and Kabanov, 2001; Koping-Hoggard et al., 2001).

The prior art contains various examples of methods for the delivery of nucleic acids to the so respiratory tract using non-viral vectors (Deshpande et al., 1998; Ferrari et al., 1997; Gautam et al., 2000). We recently identified and characterized one such system based on the DNA-complexing polymer chitosan Koping-Hoggard et al., 2001), a linear polysaccharide which can be derived from chitin. Chitosan-based gene delivery systems are also described in U.S. Pat. No. 5,972,707 (Roy et al., 1999), US Patent Application no. 2001/0031497 (Rolland et al., 2001) and in WO 98/01160.

Chitosan has been introduced as a tight junction-modifying agent for improved drug delivery across epithelial barriers (Artursson et al., 1994). It is considered to be non-toxic after oral administration to humans and has been approved as a food additive and also incorporated into a wound-healing product (Illum, 1998).

Chitosans comprise a family of water-soluble, linear polysaccharides consisting of (1→4)-linked 2-acetamido-2-deoxy-β-D-glucose (GlcNAc, A-unit) and 2-amino-2-deoxy-β-D-glucose, (GlcN, D-unit) in varying composition and sequence, confer FIG. 1. The relative content of A- and D-units may be expressed as the fraction of A-units:

F_{A}=number of A-units/(number of A-units+number of D-units)

F_{A} is related to the percentage of de-N-acetylated units through the relation:

% de-N-acetylated units=100%·(1−F_{A})

Each D-unit contains a hydrophilic and protonizable amino group, whereas each A-unit contains a hydrophobic acetyl group. The relative amounts of the two monomers (e.g. A/D=F_{A}/(1−F_{A})) can be varied over a wide range, and results in a broad variability in their chemical, physical and biological properties. This includes the properties of the chitosans in solution, in the gel state and in the solid state, as well as their interactions with other molecules, cells and other biological and non-biological matter.

The influence of the chemical structure of chitosans was recently demonstrated when chitosans were used in a non-viral gene delivery system (Koping-Hoggard et al., 2001). Chitosans of different chemical compositions displayed a structure dependent efficiency as gene delivery system. Only chitosans that formed stable complexes with pDNA gave a significant transgene expression.

Chitosans may, irrespective of their F_{A} or molecular weight, be chemically modified by introducing chemical substituents. The amino group of the glucosamine unit allows facile derivatisation due to its reactivity. Also substitution at the hydroxyl groups is a possible route to chitosan derivatives, e.g. O-carboxy methyl chitosan (Kurita, 2002).

A high number of chitosan derivatives have been described in the literature, but very few have been tested in gene delivery systems. Trimethylated chitosan has however been reported to function as gene delivery vector in epithelial cell lines (Thanou et al., 2002).

Tømmeraas et al. (2002) have described a series of branched chitosans where branching occurred by reacting aldehydes to the amino group of D-units through Schiff base formation. Monosaccharides such as glucose, galactose, disaccharides such as lactose, as well as oligosaccharides in general may be linked to chitosans through Schiff base formation between the aldehyde group of the saccharides and the unsubstituted amino groups of the chitosan as described by Yalpani & Hall (1984). In most carbohydrates the aldehyde group at the reducing end is involved in intramolecular ring formation. However, due to the well-known equilibrium between the ring form (hemiacetal) and the open chain (aldehyde form) all or most carbohydrates react as aldehydes. For keto sugars such as fructose there is a corresponding equilibrium between a ring form (hemiketal) and an open chain (keto form).

Another type of carbohydrate based aldehydes are those that may be obtained by degrading long chain carbohydrates such as chitosan or heparin with nitric acid. In this reaction residues of glucosamine are deaminated to produce 2,5-anhydro-D-mannose, which has an aldehyde group, which is not involved in the traditional ring formation. Oligomers terminating in this residue may readily be linked to the amino group of chitosan or other amines by Schiff base formation (Tømmeraas et al., 2002, Hoffman et al., 1983, Casu et al., 1986).

According to the present invention it was surprisingly discovered that certain branched chitosans were more effective complexing agents with regard to gene delivery than corresponding previously known unbranched chitosans and chitosan oligomers.

### SUMMARY OF THE INVENTION

According to one aspect the present invention is directed to a composition containing:

a) a nucleic acid; and

b) a chitosan containing branching groups covalently linked to the amino groups wherein said branches are selected from the following groups; alkyl with 2 or more carbon atoms, monosaccharides, oligosaccharides or polysaccharides. The said composition comprising branched chitosans is particularly useful for delivery of nucleic acid into cells in a host tissue. According to the present invention it has unexpectedly been found that formulations comprising nucleic acid, such as plasmid DNA, and certain branched chitosans are advantageous to achieve delivery of the nucleic acid into cells of a selected tissue and to obtain in vivo expression of the desired molecules encoded for by the various nucleic acids.

In a preferred embodiment the composition of the invention comprises branches that are obtainable in a reaction between the amino groups of the chitosan and a carbonyl compound branching group to form a Schiff base according to the scheme: 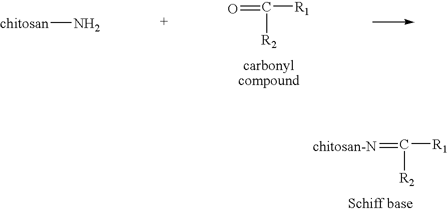
where N represents the N-atom linked to C-2 of the glucosamine residues of the chitosan, and R₁ and R₂ each independently represent a hydrogen atom, or R₁ represents a hydrogen atom and R₂ represents an optionally substituted linear or branched saturated or unsaturated hydrocarbon group having up to 10 carbon atoms, or R₁ and R₂ each independently represent an optionally substituted linear or branched saturated or unsaturated hydrocarbon group having up to 10 carbon atoms, or the carbonyl compound represents a monosaccharide, an oligosaccharide or a polysaccharide, possibly the Schiff base product is reduced to give the following type of compound:

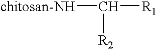

It is another object of the invention to provide a method of preparing the composition comprising nucleic acid, such as plasmid DNA, and certain branched chitosans, for delivery of nucleic acid into cells in a host tissue. The method of the invention comprising the steps of:

(a) exposing said branched chitosan of claim 1(b) to an aqueous solvent;

(b) mixing the aqueous solution of step (a) with said nucleic acid in an aqueous solvent; and

(c) reduce the volume of the product solution obtained in step (b) to achieve a desired concentration of the composition.

It is yet another object of the present invention to provide a method of administering nucleic acid, such as plasmid DNA, and certain branched chitosans, into cells in a host tissue. A method of administering a nucleic acid to a mammal, according to the present invention is by introducing the composition into the mammal.

A further object of the invention is a composition according to the invention for use as a prophylactic or therapeutic medicament in a mammal. The composition of the invention can equally be for use as an in vitro or in vivo diagnostic agent.

These and other objects of the invention are provided by one or more of the embodiments described below.

A method of preparing the composition according to the present invention, for delivery of nucleic acid into cells in a host tissue, comprises the steps of: production of certain branched chitosan, and (a) exposing said branched chitosans to an aqueous solvent in the pH range 4.0-8.0, (b) mixing the aqueous solution of step (a) with said nucleic acid in an aqueous solvent, and (c) dehydrating the solution obtained in step (b) to achieve a desired concentration of the composition before administration in vivo. Step (c) can be obtained by (1) evaporating the liquid of the product solution in step (b) to obtain the desired concentration, or (2) lyophilisate the product solution in step (b) followed by reconstitution to obtain the desired concentration.

In yet another embodiment of the invention a method for delivery of the formulation into cells in a host tissue is provided. Preferably, said composition is introduced into the mammal by administration to mucosal tissues by oral, buccal, sublingual, rectal, vaginal, nasal or pulmonary routes. According to a specific embodiment, said composition is introduced into the mammal by parenteral administration.

More specifically, the present invention is directed to a composition as defined in the claims 1-15. Further embodiments of the invention are directed to the subject matter of the claims 16-24.

Other objects, features and advantages of the present invention will become apparent from the following detailed description. It should be understood, however, that the detailed description and specific examples, while indicating preferred embodiments of the invention, are given by the way of illustration only, since various changes and modifications within the spirit and scope of the invention will become apparent to those skilled in the art from this detailed description.

### DESCRIPTION OF THE DRAWINGS

FIG. 1. Chemical structure of chitosans. In this example a fragment of a chitosan chain is shown where the fragment contains one residue of N-acetyl-β-D-glucosamine (A-unit) and 3 residues of β-D-glucosamine (1-units). The amino group of the D-units may be on a protonated or unprotonated form depending on pH.

FIG. 2. Example of a branched chitosan where branches have been introduced by reductive N-alkylation with acetaldehyde resulting in an ethyl group as a substituent on the amino group. The degree of branching can for instance be controlled by varying the amount of added acetaldehyde or by varying the reaction time.

FIG. 3. Branched chitosan where branches have been introduced by reductive N-alkylation with D-glucose.

FIG. 4. Chemical structure of a chitosan containing a residue of 2,5-anhydro-D-mannofuranose (M) located at the chain terminus corresponding to the reducing end. In this example all of the remaining residues are N-acetyl-D-glucosamine (F_{A}=1.0).

FIG. 5. Shows branching of the trimer AAM to the amino group of a chitosan by reductive amination.

FIG. 6. ¹H-NMR spectra of 4 chitosans (DPₙ=25, F_{A}<0.001) containing AAM branches with different degrees of branching (DS).

FIG. 7 shows an agarose gel retardation assay indicating the formation of stable complexes between branched chitosans and pLuc.

FIG. 8 shows the effect of branching molecule on the luciferase gene expression in 293 cells 72 h after transfection with stable complexes between branched chitosan oligomers and pLuc.

FIG. 9 shows the effect of the degree of branching with trimer on the luciferase gene expression in (A) 293 and (B) Calu-3 cells 72 h after transfection with complexes between trimer branched chitosan oligomers and pLuc.

FIG. 10 shows a time-course study of luciferase gene expression in (A) 293 and (B) Calu-3 cells after transfection with chitosan oligomers branched with 7% trimer AAM.

Using the expression of a reporter protein, luciferase, as a model for a therapeutic protein in an in vitro cell model, it was unexpectedly found that a composition according to the invention comprising plasmid DNA, and certain branched chitosans, are advantageous to achieve delivery of the nucleic acid into cells and to obtain expression of the desired molecules encoded for by the nucleic acids.

It was found that certain branched chitosans formed stable complexes, as revealed by agarose gel electrophoresis, with pLuc that resulted in high luciferase gene expression. The formation of stable complexes was found to be influenced by (1) the amine/phosphate charge ratio (+/−) between the chitosans and pDNA, (2) the degree of branching of the chitosan and, (3) the type of branching. Generally, when the degree of branching increased, a higher amine/phosphate charge ratio (+/−) between the branched chitosan and pDNA was required for the formation of stable complexes. As a result, unstable complexes mediating low gene expression, were formed even at as high charge ratio as 60:1 (+/−) with a chitosan oligomer branched with 40% timer AAM, but stable pDNA-complexes, mediating high gene expression, were formed already at charge ratio 10:1 (+/−) with the chitosan oligomer branched with 7% trimer AAM.

The fact that stable complexes resulted in a higher gene expression than unstable complexes is in agreement with the prior art (Fischer et al., 1999; Gebhart and Kabanov, 2001; Koping-Hoggard et al., 2001). Formulations with enhanced complex stabilities are thus considered advantagous with respect to in vitro gene transfection as compared to less stable complexes.

A higher luciferase gene expression was obtained with stable complexes based on said branched chitosans, as compared to unbranched chitosans.

It was found that the efficiency of mediating gene expression in the human embryonic kidney cell line 293, was dependent on the structure of the branching molecule with the following rank order: 7% trimer AAM >6% glucose >6% acetaldehyde >unbranched chitosan oligomer.

According to prior art, pDNA-complexes based on chitosan have shown a slower onset of gene expression, mediating a low gene expression at early time points as 48 h after transfection, as compared to pDNA-complexes based on the synthetic polymer polyethylenimine, PEI (Koping-Hoggard et al., 2001, Erbacher et al., 1998).

Surprisingly, a similar gene expression kinetics to PEI was obtained with pDNA-complexes based on certain chitosans branched with 7% timer AAM as compared to unbranched chitosan in the human embryonic kidney cell line 293. Also, similar kinetics of gene expression was obtained in the human lung epithelial cell line Calu-3, but unexpectedly a 10-fold higher expression was obtained with chitosan oligomers branched with 7% trimer AAM as compared to PEI.

An increased cellular uptake in airway epithelial cells, and an enhanced intracellular trafficking of pDNA complexes containing sugar residues coupled to the DNA complexing agent has been described in the prior art (Kollen et al., 1996, Fajac et al., 1999, Kollen et al., 1999). The presence of specific sugar binding lectins at the cell surface membrane but also the presence of lectins inside the cells may be responsible for the increased transfection efficiency of these pDNA systems containing sugar residues. However, in the case of e.g. polylysine having sugar residues coupled to it, the efficacy is dependent on coadministration of another agent, chloroquine, which cannot easily be targeted to the same cell as the present composition or be used in vivo due to its significant toxicity. In the above description of pDNA-complexes based on chitosans containing certain branches, no other agents were co-administrated.

Suitably, said chitosan containing branches is obtained by selecting an unbranched chitosan with F_{A} between 0 and 0.70, preferably between 0 and 0.35, more preferably between 0 and 0.10 and most preferably between 0 and 0.01. Said chitosan is then degraded by acid hydrolysis, enzymatic hydrolysis or by reaction with nitric acid to produce a weight average Degree of Polymerisation (DP_{w}) of 2-2500 preferably 3-250, and most preferably 4-50. Optionally, the degraded chitosan may be subjected to fractionation such as gel filtration to produce chitosans with more narrow molecular weight distributions. Particularly useful starting material chitosans for branching are the one described in the co pending Norwegian Patent Application no. 2002 2148, filed on even date, hereby incorporated by reference. Said chitosans are subjected to branching in a process which involves Schiff base formation so between a carbonyl compound, preferably an aldehyde, and the amino groups of D-glucosamine residues of the chitosan. The branching reaction preferably takes place in the presence of a suitable reduction agent such as NaCNBH₃ in order to reduce the Schiff bases. Generally, the degree of branching is controlled by controlling the ratio between carbonyl compound and D-glucosamine residues.

In one embodiment of the invention said carbonyl compound is acetaldehyde, which after branching with said chitosan yields the structure shown in FIG. 2.

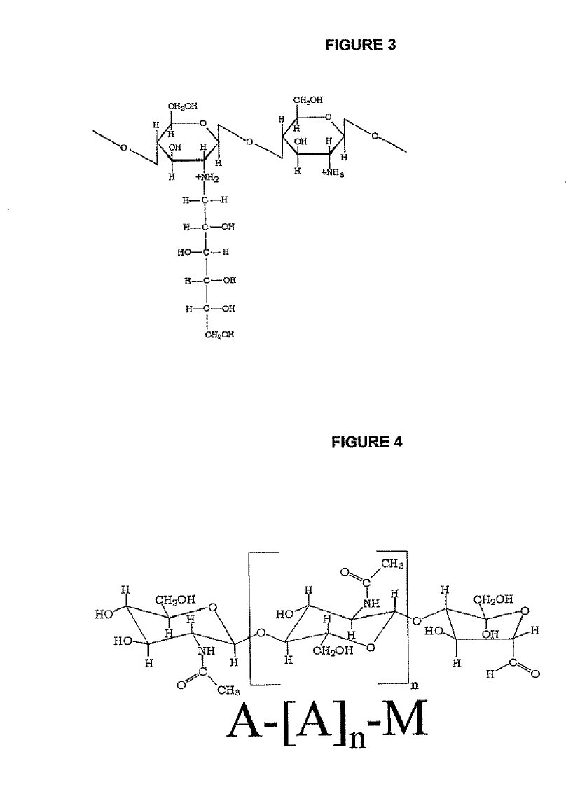

In another embodiment of the invention said carbonyl compound is D-glucose, which after branching with said chitosan yields the structure shown in FIG. 3.

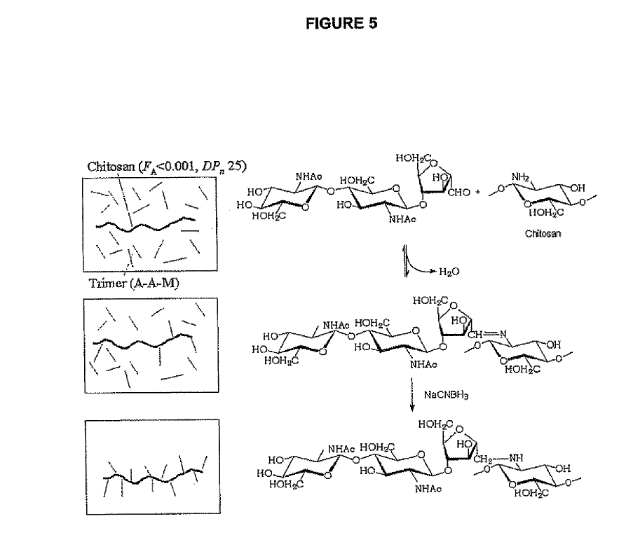

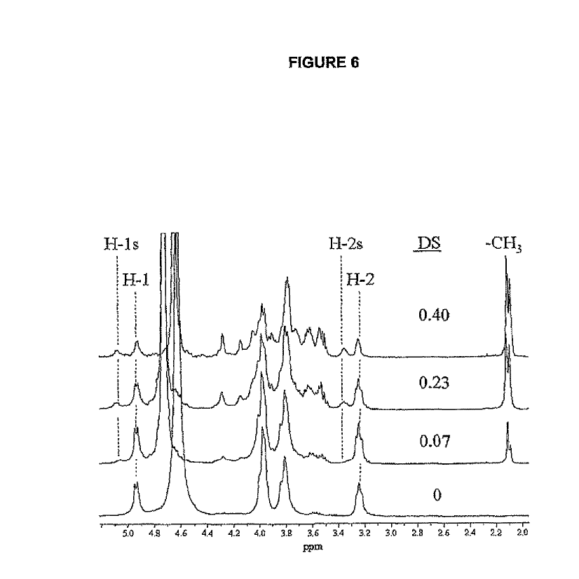

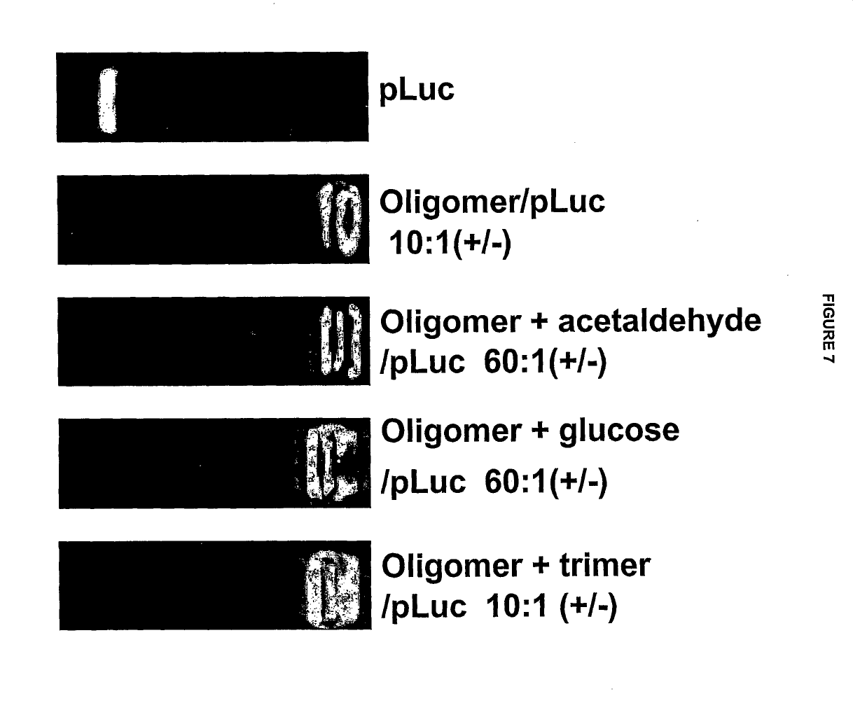

In yet another embodiment of the invention said carbonyl compound is a polysaccharide or an oligosaccharide derived from chitosan by partial depolymerisation reaction with nitric acid to obtain the desired average DP, and the reactive aldehyde 2,5-anhydro-D-mannose at the chain terminus as shown in FIG. 4 (Tømmeraas et al., 2002). Optionally, the partially degraded chitosans may be further subjected to fractionation such as gel filtration to obtain monodisperse oligomers (single DP) as described by Tømmeraas et al. (2002). These oligomers containing said reactive aldehyde may further react with any chitosan to produce branches of the type exemplified in FIG. 5.

In yet another embodiment of the invention said carbonyl compound is a polysaccharide or an oligosaccharide derived from chitosan by partial hydrolysis with acid or chitosanases to obtain the desired average DP, and a normal reducing end (Våarum et al., 2001). Optionally, the partially degraded chitosans may be further subjected to gel filtration to obtain monodisperse oligomers (single DP) as described by Tømmeraas et al., (2001). These oligomers containing said reducing ends may further react with any chitosan to produce branches as described for oligosaccharides in general by Yalpani and Hall (1984).

It should be understood, that a person skilled in the art can produce chitosan branched with other molecules such as peptides for targeting of specific tissues and/or cells and stabilizing agents such as polyethylene glycol (PEG).

The nucleic acid of the composition, of the present invention, comprises suitably a coding sequence that will express its function when said nucleic acid is introduced into a host cell.

According to another preferred embodiment of the invention, said nucleic acid is selected from the group consisting of RNA and DNA molecules. These RNA and DNA molecules can be comprised of circular molecules, linear molecules or a mixture of both. Preferably, said nucleic acid is comprised of plasmid DNA.

According to one aspect of the present invention, said nucleic acid comprises a coding sequence that encodes a biologically active product, such as a protein, polypeptide or a peptide having therapeutic, diagnostic, immunogenic, or antigenic activity.

The present invention is also concerned with compositions as described above wherein said nucleic acid comprises a coding sequence encoding a protein, an enzyme, a polypeptide antigen or a polypeptide hormone or wherein said nucleic acid comprises a nucleotide sequence that functions as an antisense molecule, such as RNA, or chemically modified RNA.

The present invention is also directed to a method for preparing the present composition, said method comprising the steps of: providing the branched chitosan as described above, (a) exposing said branched chitosan to an aqueous solvent in the pH range 3.5-8.0, (5) mixing the aqueous solution of step (a) with said nucleic acid in an aqueous solvent, and (c) dehydrating the product solution obtained in step (b) to achieve a high concentration of the composition before administration in vivo. Step (c) can be obtained by (1) evaporating the liquid of the product solution in step (b) to obtain the desired concentration, or (2) lyophilizate the product solution in step (b) followed by reconstitution to obtain the desired concentration. Typically, the said nucleic acid is present at a concentration of 1 ng/ml-3000 μml, preferably 1 μg/ml-100 μg/ml and most preferably 10-50 μg/ml in step (b) and 10 μg/ml-3,000 μg/ml, preferably 10 μg/ml-1,000 μg/ml and most preferably 100-500 μg/ml in step (c) (1).

It should be understood, that a person skilled in the art can form the present composition at different amine/phosphate charge ratios to include negative, neutral or positive charge ratios.

The present invention is further concerned with a method of administering nucleic acid to a mammal, using the composition of the present invention, and introducing the composition into the mammal. Preferably, said composition is introduced into the mammal by administration to mucosal tissues by pulmonary, nasal, oral, buccal, sublingual, rectal or vaginal routes. According to a specific embodiment, said composition is introduced into the mammal by parenteral administration.

The present invention is also concerned with use of the composition described above in the manufacture of a medicament for prophylactic or therapeutic treatment of a mammal or in the manufacture of a diagnostic agent for in vivo or in vitro diagnostic methods, and specifically in the manufacture of a medicament for use in gene therapy, antisense therapy or genetic vaccination for prophylactic or therapeutic treatment of malignancies, autoimmune diseases, inherited disorders, pathogenic infections and other pathological conditions.

### EXAMPLES

### Example 1

### Preparation of Fully de-N-acetylated Chitosan (F_{A}<0.01)

Commercially available chitosan with F_{A} of 1.0 (10 g) was further de-N-acetylated by heterogeneous alkaline deacetylation (50% (w/w) NaOH solution for 4 hours at 100° C. in an airtight glass-container). The chitosan was filtered and washed with 2×150 mL of methanol and 1×150 mL of methyl ether before drying over night at room temperature, followed by subsequent dialysis against 0.2 M NaCl and deionised water. ¹H NMR spectroscopy showed that F_{A}<0.01

### Example 2

### Depolymerisation of Fully de-N-acetylated Chitosan (DPₙ=25)

Chitosan (F_{A}<0.01, 500 mg in HCl form) was depolymerised by nitrous acid (17 mg NaNO₂) as described by Allan and Peyron (1989, 1995a,b), followed by conventional reduction by NaBH₄, dialysis and lyophilisation. The chitosan was found to be fully reduced and the average number degree of polymerisation (DPₙ) was determined to 25 by ¹H and ¹³C NMR spectroscopy.

### Example 3

### Preparation of N-Acetylated Oligomers with a Reactive Reducing End

Chitosan (F_{A}=0.59, intrinsic viscosity [η]=826 mL/g, 500 mg, HCl form) was dissolved in 30 mL 2.5% v/v acetic acid. Dissolved oxygen was removed by bubbling nitrogen gas through the solution for 5 minutes. After cooling to 4° C., a freshly prepared solution of NaNO₂ (100 mg) was added, and the reaction was allowed to proceed for 12 hours at 4° C. in darkness. The product was centrifuged (10 minutes, 5000 rpm) and filtrated (8 μm), to remove the insoluble fractions of fully N-acetylated oligomers before lyophilisation.

### Example 4

### Separation of the N-acetylated Oligomers and Determination of their Chemical Structures

The oligomers (500 mg) were separated by gel filtration on two 2.5 cm×100 cm columns connected in series packed with Superdex 30 (Pharmacia Biotech, Uppsala), eluted with 0.15 M ammonium acetate at pH 4.5 at a flow rate of 0.8 mL/min. The elution was monitored by means of an on-line refraction index (RI) detector (Shimadzu RID-6A). Fractions of 4 mL were collected and pooled to provide the purified oligomers after a final lyophilisation step.

### Example 5

### Preparation of Fully de-N-Acetylated Chitosans Branched with Oligosaccharides

Fully de-N-acetylated chitosan (F_{A}<0.001, DPₙ=25) was reductively N-alkylated by purified trimer after the following procedure: A solution of low molecular-weight fully de-N-acetylated chitosan (DPₙ=25, 20 mmol D-units) and fully N-acetylated trimer (A-A-M) (2.0, 12, 20 and 40 μmol) in 0.1 M acetic acid with 0.1 M NaCl was allowed to react for four days (5 mL, pH 5.5, room temperature). NaCNBH₁₃ (50 mg) was added to the reaction mixture after 2 and 24 hours, respectively. The pH during the reaction never exceeded 6.5. Remaining not reacted trimer (A-A-M) was removed by dialysis, and the branched chitosans were converted so to the chloride salts, lyophilised and stored at −20° C.

### Example 6

### Preparation of Fully de-N-acetylated Chitosans Branched with D-Glucose

Fully de-N-acetylated chitosan (F_{A}<0.01, DPₙ=25) was reductively N-alkylated with D-glucose by the same procedure as described in Example 5, the only difference is that trimer (A-A-M) is replaced with D-glucose (4.0 μmol).

### Example 7

### Preparation of Fully de-N-acetylated Chitosans Branched with Acetaldehyde

Fully de-N-acetylated chitosan (F_{A}<0.017, DPₙ=25) was reductively N-alkylated by acetaldehyde by the same procedure as described in Example 5, the only difference is that trimer (A-A-M) is replaced with acetaldehyde (4.0 μmol).

### Example 8

### Formulation of a Composition Containing Branched Chitosan and pDNA

Chitosan oligomers and chitosan oligomers branched with 6, 10 and 20% acetaldehyde and glucose, respectively, and with 7, 23 and 40% of the trimer AAM were prepared from chitosan according to the methods described in Examples 5 to 7. Firefly luciferase plasmid DNA (pLuc) was purchased from Aldevron, Fargo, N. Dak., USA. Stock solutions of cationic chitosan oligomers (2 mg/ml) were prepared in sterile distilled deionized water, pH 6.2±0.1 followed by sterile filtration. Complexes between cationic chitosan oligomers and pLuc were formulated at charge ratios of 10:1, 30:1 and 60:1 (+/−) by adding cationic oligomer and then pLuc to sterile water under intense stirring on a vortex mixer (Heidolph REAX 2000, KEBO Lab, Spånga, Sweden). The concentration of pDNA was kept constant at 13.3 μg/ml. In addition, pLuc was formulated with PEI 251kDa (Aldrich Sweden, Stockholm, Sweden) at a previously optimized charge ratio of 5:1(+/−) (Bragonzi et al., 2000; Koping-Hoggard et al., 2001).

The complexes were tested for stability in the agarose gel electrophoresis assay. The stability of the complexes was highly dependent on the degree of branching. No stable complexes were formed with the chitosan oligomers branched with acetaldehyde and glucose at 10 and 20% degree of branching. Neither did the chitosan oligomer branched with 40% trimer form stable complexes in this assay.

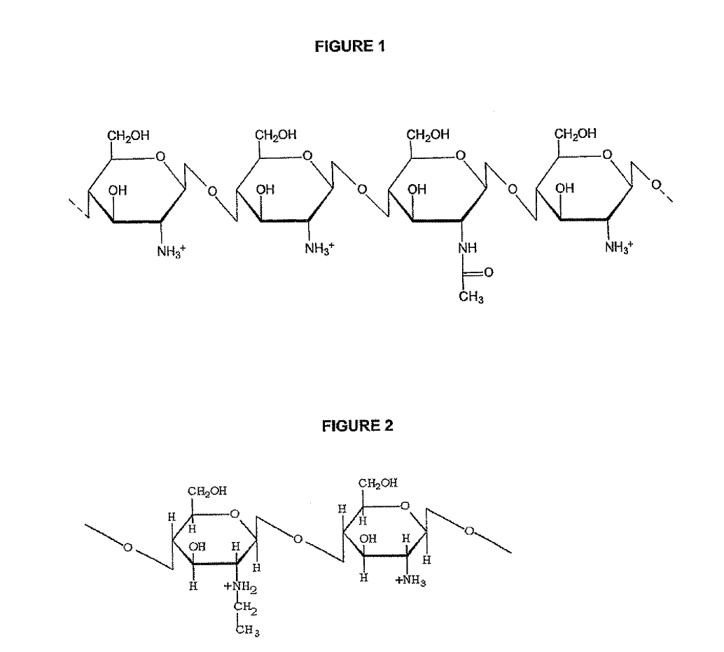

FIG. 7 shows an agarose gel retardation assay indicating the formation of stable complexes between branched chitosan oligomers and pLuc. The unsubstituted chitosan oligomers and the chitosan branched with 7% trimer AAM formed stable complexes with pDNA already at a charge ratio of 10:1 (+/−) (FIG. 1). However, as high charge ratio as 60:1 (+/−) was required for the formation of stable complexes with the chitosan oligomers branched with 6% acetaldehyde and glucose, respectively.

### Example 9

### Gene Expression Studies with Formulations Containing Branched Chitosan Oligomers and pDNA

Complexes between branched chitosan oligomers and pLuc were prepared as described in Example 8. 24 h before transfection, the epithelial human embryonic kidney cell line 293 (ATCC, Rockville, Md., USA) was seeded at 70% confluence in 96-well tissue culture plates (Costar, Cambridge, UK). The human epithelial lung cell line Calu-3 was seeded at 100,000 cells/cm² in 96-well tissue culture plates (Costar) and were cultured for 14 days to obtain differentiated cells before transfection. Prior to transfection, the cells were washed and then 50 μl (corresponding to 0.33 μg pLuc) of the complex formulations was added per well. After 5 h incubation, the formulations were removed and 0.2 ml of fresh culture medium was added. The medium was changed every second day for experiments exceeding two days. At indicated time points, cells were washed with PBS pH 7.4), lysed with Lysis buffer (Promega, Madison, Wis.) and luciferase gene expression was measured with a luminometer (Mediators PhL, Vienna, Austria). The amount of luciferase expressed was determined from a standard curve prepared with firefly luciferase (Sigma, St. Louise, Mo.). The total protein content in each sample was analyzed by the BCA assay (Pierce, Rockford, Ill.) and quantified using BSA (bovine serum albumin) as a reference protein. The absorbance was measured at 540 nm on a microplate reader (Multiscan MCC/340, Labsystems Oy, Helsinki, Finland). Luciferase gene expression (pg luciferase/μg total cell protein) is reported as mean values ±one standard, n=3-6.

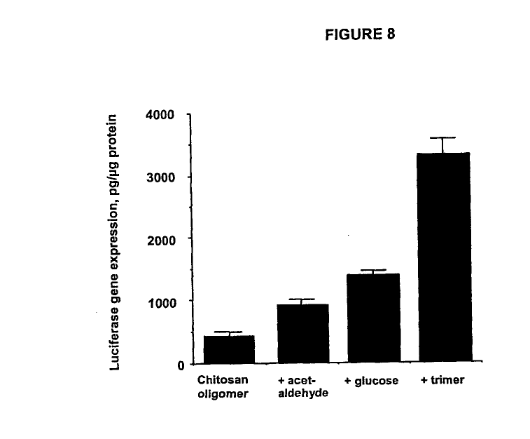

FIG. 8 shows the effect of branching molecule on the luciferase gene expression in 293 cells 72 h after transfection with complexes between branched chitosan oligomers and pLuc. The rank order of transfection efficiency was 7% timer AAM>6% glucose >6% acetaldehyde >unbranched chitosan oligomer.

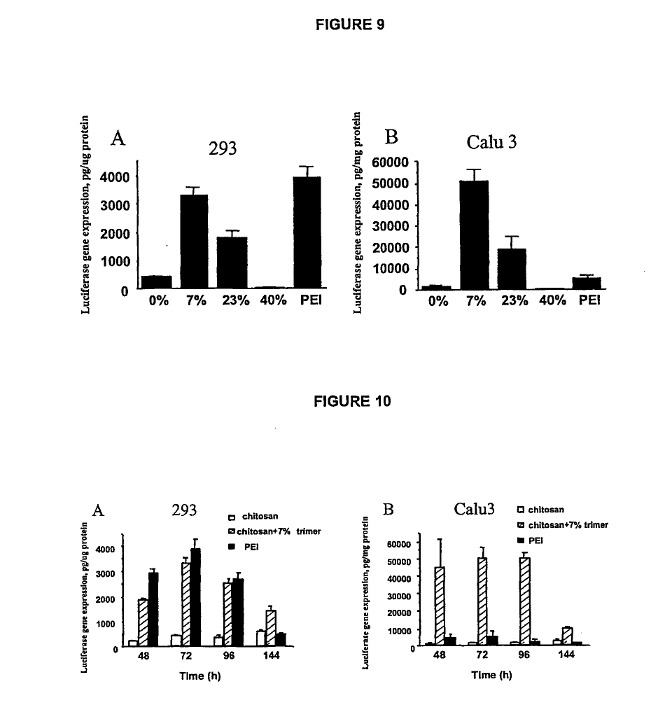

FIG. 9 shows the effect of the degree of branching with trimer AAM on the luciferase gene expression in (A) 293 and (B) Calu-3 cells 72 h after transfection with complexes between trimer branched chitosan oligomers and pLuc. In 293 cells, the rank order of efficiency was: PEI≈7% trimer>23% timer>unbranched chitosan oligomer>40% timer. Surprisingly, in the Calu-3 cell line another rank order of efficiency was obtained: 7% trimer>23% timer>PEI>unbranched chitosan oligomer>40% trimer. The low transfection efficiency obtained with the oligomer with 40% degree of branching can be explained by that unstable complexes were formed at this high degree of branching.

FIG. 10 shows a time-course study of luciferase gene expression in (A) 293 and (B) Calu-3 cells after transfection with chitosan oligomers branched with 7% timer AAM. Surprisingly, in the 293 cell line, a fast onset of gene expression, comparable to PEI, was observed with pLuc complexes based on chitosan oligomers branched with 7% timer AAM. Also, in the Calu-3 cell line, chitosan oligomers branched with 7% trimer AAM mediated a 10-fold higher luciferase gene expression compared to PEI.

### REFERENCES

Artursson P, Lindmark T, Davis S S and Illum L (1994) Effect of chitosan on the permeability of monolayers of intestinal epithelial cells (Caco-2). Pharm Res 11:1358-1361.
Boussif O, Lezoualch F, Zanta M A, Mergny M D, Scherman D, Demeneix B and Behr J P (1995) A versatile vector for gene and oligonucleotide transfer into cells in culture and in vivo: Polyetylenimine. Proceedings of the National Academy of Sciences USA 92:7297-7301.
Bragonzi A, Dina G, Villa A, Calori G, Biffi A, Bordignon C, Assael B M and Conese M (2000) Biodistribution and transgene expression with nonviral cationic vector/DNA complexes in the lungs. Gene Ther 7:1753-1760.
Casu B, Colombo M, Compagnoni T, Naggi A, Pivari E, Torri G (986) Branched chitosans with N-acetyl-glucosamine oligosaccharide side-chains, in R. A. A. Muzzarelli, L. Jeauniaux, G. W. Gooday (Eds.) Chitin in Nature & Technology, Plenum Press, New York, 1986, pp. 309-315.
Deshpande D, Blezinger P, Pillai R, Duguid J, Freimark B and Rolland A (1998) Target specific optimization of cationic lipid-based systems for pulmonary gene therapy. Pharm Res 15:1340-1347.
Erbacher P, Zou S, Bettinger T, Steffan A M and Remy J S (1998) Chitosan-based vector/DNA complexes for gene delivery: biophysical characteristics and transfection ability. Pharmaceutical Research 15:1332-1339.
Fajac I, Briand P, Monsigny M and Midoux P (1999). Sugar-mediated uptake of glycosylated polylysines and gene transfer into normal and cystic fibrosis airway epithelial cells, Human Gene Therapy 10:395-406.
Felgner J H, Kumar R, Sridhar C N, Wheeler C Y, Tsai Y J, Border R, Ramsey P, Martin M and Felgner P L (1994) Enhanced gene delivery and mechanism studies with a novel series of cationic lipid formulations. Journal of biological Chemistry 269:255-2561.
Ferrari S, Moro E, Pettenazzo A, Behr J, Zacchello F and Scarpa M (1997) ExGen 500 is an efficient vector for gene delivery to lung epithelial cells in vitro and in vivo. Gene Therapy 4:1100-1106.
Fischer D, Bieber T, Li Y, Elsasser H P and Kissel T (1999) A novel non-viral vector for DNA delivery based on low molecular weight, branched polyethylenimine: effect of molecular weight on transfection efficiency and cytoxicity. Pharm Res 16:1273-1279.
Florea B, Thanou, M., Geldof, M., Meaney, C., Junginger, H. B. and Borchard, G. (2001) Modified chitosan oligosaccharides and polyethylenimine as transfection agents for gene therapy in cystic fibrosis. Journal
Gautam A, Densmore C L, Golunski E, Xu B and Waldrep J C (2001) Transgene expression in mouse airway epitheliun by aerosol gene therapy with PEI-DNA complexes. Mol Ther 3:551-556.
Gautam A, Densmore C L, Xu B and Waldrep J C (2000) Enhanced gene expression in mouse lung after PEI-DNA aerosol delivery. Mol Ther 2:63-70.
Gebhart C L and Kabanov A V (2001) Evaluation of polyplexes as gene transfer agents. J Control Release 73:401-416.
Godbey W T, Wu K K and Mikos A G (1999) Size matters: Molecular weight affects the efficiency of poly(ethylenimine) as a gene delivery vehicle. J Biomed Mater Res 45:268-275.
Godbey W T, Wu K K and Mikos A G (2001) Poly(ethylenimine)-mediated gene delivery affects endothelial cell function and viability. Biomaterials 22:471-480.
Griesenbach U, Chonn A, Cassady R, Hannam V, Ackerley C, Post M, Tanswell A K, Olek K, O'Brodovich H and Tsui L C (1998) Comparison between intratracheal and intravenous administration of liposome-DNA complexes for cystic fibrosis lung gene therapy. Gene Ther 5:181-188.
Hoffman J, Larm O, Scholander E (1983). A new method for covalent coupling of heparin and other glycosaminoglycans to substances containing primary amino groups. Carbobydr. Res. 117:328-331.
Hudde T, Rayner S A, Comer R M, Weber M, Isaacs J D, Waldmann H, Larkin D F and George A J (1999) Activated polyamidoamine dendrimers, a non-viral vector for gene transfer to the corneal endothelium. Gene Ther 6:939-943.
Illumn L (1998) Chitosan and its use as a pharmaceutical excipient. Pharm Rev 15:1326-1331.
Kollen W, Midoux P, Erbacher P, Yip A, Roche A C, Monsigny M, Glick M C and Scanlin T (1996) Gluconoylated and glycosylated polylysine as vectors for gene transfer into cystic fibrosis airway epithelial cells. Human Gene Therapy 7:1577-1586.
Kollen W, Mulberg A E, Wei X, Sugita M, Raghuram V, Wang J, Foskett J, Glick M and Scanlin T (1999). High-efficiency transfer of cystic fibrosis transmembrane conductance regulator cDNA into cystic fibrosis airway cells in culture using lactosylated polylysine as a vector. Human Gene Therapy 10:615-622.
Koping-Hoggard M, Melnikova, Y., Vårum K. M., Lindman, B. and Artursson, P. (2001) Chitosan polyplexes as a gene delivery system. Characterization of structure-activity relationships from supramolecular shape. Submitted
Koping-Hoggard M, Tubulekas I, Guan H, Edwards K, Nilsson M, Vårum K and Artursson P (2001) Chitosan as a nonviral gene delivery system. Structure-property relationships and characteristics compared with polyethylenimine in vitro and after lung administration in vivo. Gene Ther 8:1108-1121.
Kurita K. (2001) Controlled functionalization of the polysaccharide chitin Prog. Polym. Sci. 26:1921-1971.
Ledley F D (1996) Pharmaceutical approach to somatic gene therapy. Pharm Res 13:1595-1614.
Li S and Huang L (1997) In vivo gene transfer via intravenous administration of cationic lipid-protamine-DNA (LPD) complexes. Gene Ther 4:891-900.
Li S, Tan Y, Viroonchatapan E, Pitt B R and Huang L (2000) Targeted gene delivery to pulmonary endothelium by anti-PECAM antibody. Am J Physiol Lung Cell Mol Physiol 278:L504-511.
Luo D and Saltzman W M (2000) Synthetic DNA delivery systems. Nat Biotechnol 18:33-37.
MacLaughlin F C, Mumper R J, Wang J, Tagliaferri J M, Gill I, Hinchcliffe M and Rolland A P (1998) Chitosan and depolymerized chitosan oligomers as condensing carriers for in vivo plastid delivery [In Process Citation]. J Controlled Release 56:259-272.
Mumper R J, Duguid J G, Anwer K, Barton M K, Nitta H and Rolland A P (1996) Polyvinyl derivatives as novel interactive polymers for controlled gene delivery to muscle. Pharm Res 13:701-709.
Mumper R J, Wang J, Klakanp S L, Nitta H, Anwer K, Tagliaferri F and Rolland A P (1998) Protective interactive noncondensing (PINC) polymers for enhanced plasmid distribution and expression in rat skeletal muscle. J Control Release 52:191-203.
Ottøy (1996) Carbohydrate Polymers 31:253-261
Plank C, Tang M X, Wolfe A R and Szoka F C, Jr (1999) Branched cationic peptides for gene delivery: role of type and number of cationic residues in formation and in vitro activity of DNA polyplexes [published erratum appears in Hum Gene Ther 1999 Sep. 1; 10(133:2272]. Hum Gene Ther 10:319-332.
Putnam D, Gentry C A, Pack D W and Langer R (2001) Polymer-based gene delivery with low cytotoxicity by a unique balance of side-chain termini. Proc Nat Acad Sci USA 98:1200-1205.
Roy K, Mao H Q, Huang S K and Leong K W (1999) Oral gene delivery with chitosan-DNA nanoparticles generates immunologic protection in a murine model of peanut allergy. Nat Med 4:387-391.
Saeki Y, Matsumoto N, Nakano Y, Mori M, Awai K and Kaneda Y (1997) Development and characterization of cationic liposomes conjugated with HVJ (Sendai virus): reciprocal effect of cationic lipid for in vitro and in vivo gene transfer. Hum Gene Ther 8:2133-2141.
Sato T, Ishii T and Okahata Y (2001) In vitro gene delivery mediated by chitosan. effect of pH, serum, and molecular mass of chitosan on the transfection efficiency. Biomaterials 22:2075-2080.
Song Y K, Liu F, Chu S and Liu D (1997) Characterization of cationic liposome-mediated gene transfer in vivo by intravenous administration. Hum Gene Ther 8: 1585-1594.
C. Tanford (1961) Physical chemistry of macromolecules, John Wiley and Sons, New York Section 8b and 33a)
Thanou M, Florea B I, Geldof M, Junginger B E, Borchard G. (2002). Quarternized chitosan oligomers as novel gene delivery vectors in epithelial cell lines. Biomaterials 23:153-159.
Tømmeraas K, Vårum KM, Christensen B E and Smidsrød O (2001) Preparation and characterisation of oligosaccharides produced by nitrous acid depolymerisation of chitosans. Carbohydr Res 333:137-144.
Tømmeraas, K, M. Köping-Höggärd, K. M. Vårum, BE. Christensen, P. Artursson, and O. Smidsrød (2002) Preparation and characterisation of chitosans with oligosaccharide branches. Submitted Carbohydrate Research.
Vårum K M, Anthonsen M W, Grasdalen H and Smidsrød O (1991) Determination of the degree of N-acetylation and the distribution of N-acetyl groups in partially N-deacetylated chitins (chitosans) by high-field n.m.r. spectroscopy. Carbohydr Res 211:17-23.
Vårum, KM, Ottøy, MH and Smidsrød, O (2001) Acid hydrolysis of chitosans. Carbohydr. Polym. 46: 89-98.
Wolff J A, Malone R W, Williams P, Chong W, Acsadi G, Jani A and Felgner P L (1990) Direct gene transfer into mouse muscle in vivo. Science 247:1465-1468.
Yalpani M and Hall D H (1984). Some chemical and analytical aspects of polysaccharide modifications. 3. Formation of branched-chain, soluble chitosan derivatives. Macromolecules 17:272-281.

1: A composition containing:
a) a nucleic acid; and
b) a chitosan containing branching groups covalently linked to the amino groups wherein said branches are selected from the following groups; alkyl with 2 or more carbon atoms, monosaccharides, oligosaccharides or polysaccharides.

2: The composition of claim 1, wherein the fraction of N-acetyl-D-glucosamine residues (F_{A}) of said chitosan between 0 and 0.70, preferably between 0 and 0.35, more preferably between 1 and 0.10, and most preferably between 0 and 0.01.

3: The composition of claim 1, wherein the weight average Degree of polymerisation (DP_{w}) of said chitosan is 2-2500, preferably 3-250, and most preferably 4-50.

4: The composition of claim 1, where 1-60% of the D-glucosamine residues of said chitosan carry branching groups, preferably 2-40%, and most preferably 3-20%.

5: The composition of claim 1, wherein said branches are obtainable in a reaction between said amino groups and a carbonyl compound branching group to form a Schiff base according to the scheme: 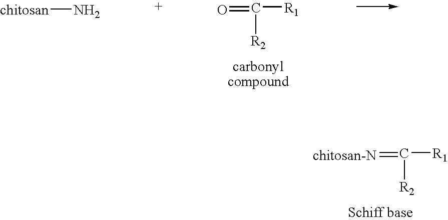
where N represents the N-atom linked to C-2 of the glucosamine residues of the chitosan, and R₁ and R₂ each independently represent a hydrogen atom, or R₁ represents a hydrogen atom and R₂ represents an optionally substituted linear or branched saturated or unsaturated hydrocarbon group having up to 10 carbon atoms, or R₁ and R₂ each independently represent an optionally substituted linear or branched saturated or unsaturated hydrocarbon group having up to 10 carbon atoms, or the carbonyl compound represents a monosaccharide, an oligosaccharide or a polysaccharide, possibly the Schiff base product is reduced to give the following type of compound:

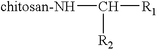

6: The composition of claim 5, wherein said carbonyl compound is acetaldehyde where R₁ represents a hydrogen atom, and R₂ represents an ethyl group.

7: The composition of claim 5, wherein said carbonyl compound is the monosaccharide D-glucose.

8: The composition of claim 5, wherein said carbonyl compound is an oligomer consisting of 1→4 linked residues of D-glucosamine with a residue of 2,5-anhydro-D-mannose at the reducing end according to the formula: 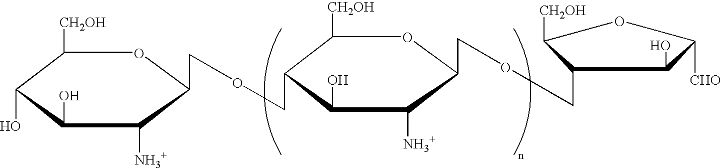
where n represents the number of non-terminal residues and is between 0-100, preferably 0-10 and most preferably 0-3, F_{A} of the oligomer is optionally in the range 0-0.5.

9: The composition of claim 5, wherein said carbonyl compound is an oligosaccharide consisting of 1→4 linked residues of N-acetyl-D-glucosamine with a residue of 2,5-anhydro-D-mannose at the reducing end according to the formula: 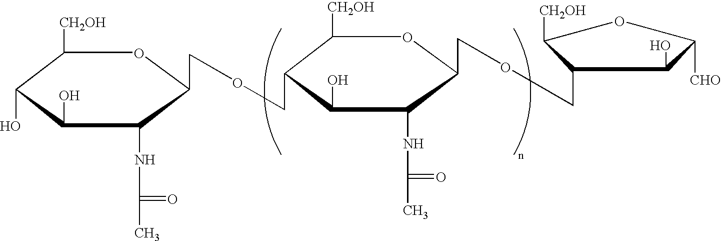
where n represents the number of non-terminal residues and is between 0-100, preferably 0-10, and most preferably between 0-3.

10: The composition of claim 5, wherein said carbonyl compound is an oligomer consisting of 1→4 linked residues of N-acetyl-D-glucosamine according to the formula 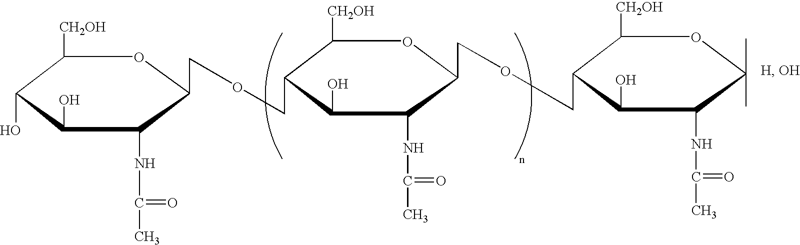
wherein H, OH represents the α- or β-anomer of the reducing end, and n represents the number of non-terminal residues and is between 0-100, preferably 0-10, and most preferably between 0-3.

11: The composition of claim 5, wherein said carbonyl compound is an oligomer consisting of 1→4 linked residues of D-glucosamine according to the formula: 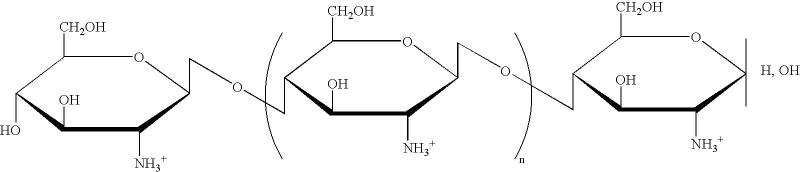
wherein H, OH represents the α- or β-anomer of the reducing end, and n represents the number of non-terminal residues and is between 0-100, preferably 0-10, and most preferably between 0-3, F_{A} of the oligomers is optionally in the range 0-0.5.

12: The composition of claim 1, wherein said composition essentially has a net positive charge ratio.

13: The composition of claim 1, wherein said composition has a pH in the range of 3.5 to 8.0.

14: The composition of claim 1, wherein said nucleic acid comprises a coding sequence that will express its function when said nucleic acid is introduced into a host cell.

15: The composition of claim 1, wherein said nucleic acid is selected from the group consisting of DNA and RNA molecules.

16: A method of preparing a composition of claim 1, comprising the steps of:
(a) exposing the branched chitosan of claim 1 (b) to an aqueous solvent;
(b) mixing the aqueous solution of step (a) with said nucleic acid in an aqueous solvent; and
(c) reducing the volume of the product solution obtained in step (b) to achieve a desired concentration of the composition.

17: A method of administering a nucleic acid to a mammal, using the composition according to claim 1, by introducing the composition into the mammal.

18: The method of claim 17, wherein the composition is administrated to the mammal by introduction onto mucosal tissues by pulmonary, nasal, oral, buccal, sublingual, rectal or vaginal routes.

19: The method of claim 17, wherein the composition is administrated to the mammal by introduction into submucosal tissues by parenteral routes that is; intravenous, intramuscular, intradermal, subcutaneous or intracardiac administration, or to internal organs, blood vessels or other body surfaces or cavities exposed during surgery.

20: The method of claim 17, comprising the composition of claim 1, whereby said nucleic acid is capable of expressing its function inside said mammal.

21: The composition of claim 1, for use as a prophylactic or therapeutic medicament in a mammal.

22: The composition of claim 21, for the use in gene therapy, antisense therapy or genetic vaccination for prophylactic or therapeutic treatment of malignancies, autoimmune diseases, inherited disorders, pathogenic infections and other pathological diseases.

23: The composition of claim 1, for use as an in vitro or in vivo diagnostic agent.
